(19) United States
(12) Patent Application Publication
(10) Pub. No.: US 2005/0058644 A1
(43) Pub. Date: Mar. 17, 2005

(54) Combination therapy of gamma-interferon and B-cell specific antibodies

(75) Inventor: Edgar Engleman, Palo Alto, CA (US)

Correspondence Address: MERCHANT & GOULD PC, P.O. BOX 2903, MINNEAPOLIS, MN, 55402-0903, US

(73) Assignee: INTERMUNE, INC., Brisbane, CA (US)

(21) Appl. No.: 10/730,856

(22) Filed: Dec. 8, 2003

Related U.S. Application Data
(63) Continuation of application No. PCT/US02/19023, filed on Jun. 14, 2002.
(60) Provisional application No. 60/298,354, filed on Jun. 14, 2001.

(51) Int. Cl. A61K 39/395

(52) U.S. Cl. 424/145.1; 424/141.1

(57) ABSTRACT

A combination therapy is provided of IFN-γ and therapeutic antibodies that recognize determinants on hyperproliferative B lineage cells. The targeted cells are contacted with these agents either locally or systematically, usually systematically. The subject methods provide a means for therapeutic treatment and investigation of hyperproliferative disorders.

This application is being filed as a PCT international patent application in the name of InterMune, Inc., a U.S. national corporation (applicant for all countries except the U.S.), and Ed Engleman, a U.S. citizen (applicant for U.S. designation), on 14 Jun. 2002, designating all countries.

### BACKGROUND OF THE INVENTION

B cell neoplasias represent a heterogeneous group of diseases, including acute lymphocytic leukemia (ALL) and the broad spectrum of non-Hodgkin's lymphomas (NHL). These diseases are significant contributors to cancer mortality. Responses to therapy are mixed, and there are significant numbers of patients that either do not respond initially, or that relapse after currently available treatment. Traditional methods of treating B cell malignancies, including chemotherapy and radiotherapy, have limited utility due to toxic side effects.

For example, the majority of chronic lymphocytic leukemias are of B cell lineage. This type of B cell malignancy is the most common leukemia in the Western world. In the early phases of the disease it is indolent, characterized by the accumulation of small mature functionally-incompetent malignant B cells having a lengthened life span. Eventually, the doubling time of the malignant B cells decreases and patients become increasingly symptomatic. While treatment can provide symptomatic relief, the overall survival of the patients is only minimally affected. The late stages of chronic lymphocytic leukemia are characterized by significant anemia and/or thrombocytopenia. At this point, the median survival is less than two years. Due to the very low rate of cellular proliferation, chronic lymphocytic leukemia is resistant to treatment.

Recently there has been substantial interest in the use of antibodies as therapeutic agents, in order to provide a targeted treatment. This enthusiasm has stemmed from advances in recombinant technology allowing for the production of chimeric and humanized antibodies, from Food and Drug Administration approval of radioimmunoconjugates for use in diagnosis and staging in colorectal, ovarian, and prostate cancer, from studies demonstrating durable response rates in lymphoma and breast cancer, and from trials demonstrating marked efficacy of radiolabeled antibodies in the treatment of non-Hodgkin's lymphoma.

Unconjugated monoclonal antibodies investigated for the treatment of hematologic malignancies include anti-idiotype, CAMPATH-1, and rituximab. Rituximab was the first such therapy approved in the United States for relapsed or refractory low-grade or follicular B-cell non-Hodgkin's lymphoma after demonstration of an overall response rate of 48% and a duration of response of 11.7 months.

Rituximab is a chimeric anti CD-20 monoclonal antibody containing human IgG1 kappa constant regions, with murine variable regions. The anti-lymphoma effects of Rituximab are probably due to complement and antibody-dependent cell-mediated cytotoxicity, and induction of apoptosis. Phase II trials have demonstrated a strong activity of rituximab alone in indolent B cell non-Hodgkin lymphoma, especially in patients with follicular lymphoma. More recently, Rituximab has shown activity also in diffuse large cell lymphoma, mantle cell lymphoma and in other B-malignancies. Good results have also been obtained utilizing Rituximab for in vivo purging. However, we are still far from having found a definite position for Rituximab in the treatment of lymphoproliferative disorders.

A need exists to develop an immunotherapy for B-cell malignancies that allows repeated administration of comparatively low doses of an antibody, and that is not limited by the necessity of adding a toxic agent for achieving a therapeutic response of significant duration.

Relevant Literature

A review of the use of Rituximab may be found in Hainsworth (2000) The Oncologist 5:376-384. Descriptions of treatment are provided by Byrd et al. (2000) J. Clin. Oncol. 19:2153-2164; McLaughlin et al. (2000) Semin. Oncol. 27:37-41; Davis et al. (2000) Clin. Cancer R s. 6:2644-2652. The treatment of plasma cell dyscracias with Rituxan is described by Treon et al. (1999) Semin. Oncol. 26(5 Suppl 14):97-106.

Patents of interest include U.S. Pat. Nos. 6,171,586; 5,776,456; 6,224,866; and 6,183,744.

### SUMMARY OF THE INVENTION

Compositions and methods are provided for the use of B cell specific antibodies in combination with gamma-interferon (IFN-γ), as a combination therapy to treat B cell hyperproliferative disorders. The targeted cells are contacted with these compounds either locally or systemically. Synergistic combinations may provide for comparable or improved therapeutic effects, for example an increased response rate (defined as partial or complete response), an increased time to recurrence, or increased time to disease progression, while potentially lowering adverse side effects.

### DETAILED DESCRIPTION OF THE EMBODIMENTS

Therapeutic antibodies that recognize determinants on hyperproliferative B lineage cells, e.g. leukemias and lymphomas of B cell origin; are administered in combination with IFN-γ as a combination therapy. Although the antibodies can provide some benefit and when administered alone, the immunomodulatory effect of the IFN-γ enhances the therapy. Antibodies of particular interest include anti-CD20 antibodies, e.g. rituxan, which has been shown to be effective in the treatment of B cell malignancies. The targeted cells are contacted with these agents either locally or systemically, usually systemically. The subject methods provide a means for therapeutic treatment and investigation of hyperproliferative disorders. Animal models, particularly small mammals, e.g. murine, lagomorpha, etc. are of interest for experimental investigations.

The subject methods are used for prophylactic or therapeutic purposes. The term “treatment” as used herein refers to reducing or alleviating symptoms in a subject, preventing symptoms from worsening or progressing, inhibition or elimination of the causative agent, or prevention of the disorder in a subject who is free therefrom. For example, treatment of a cancer patient may result in reduction of tumor size, elimination of malignant cells, prevention of metastasis, or the prevention of relapse in a patient who has been initially responsive to therapy. The treatment of ongoing disease, to stabilize or improve the clinical symptoms of the patient, is of particular interest. Such treatment is desirably performed prior to complete loss of function in the affected tissues.

It is believed that the anti-proliferative effects of antibodies that recognize tumor antigens are due to complement and antibody-dependent cell-mediated cytotoxicity (ADCC), and induction of apoptosis. The methods of the invention may enhance these killing mechanisms. For example, IFN-γ is known to increase expression of Fc receptors on both blood monocytes and polymorphonuclear cells, e.g. neutrophils and eosinophils, which cells are involved in ADCC. In a preferred embodiment, the IFN-γ is administered prior to administration of the antibodies, in order to initiate immunomodulatory activity. Other immunomodulatory agents that enhance ADCC may also be administered in combination with the IFN-γ, e.g. G-CSF, GM-CSF; IL-10 and TGF-β.

### DEFINITION OF TERMS

The present invention provides novel compositions and methods as set forth within this specification. In general, all technical and scientific terms used herein have the same meaning as commonly understood to one of ordinary skill in the art to which this invention belongs, unless clearly indicated otherwise. For clarification, listed below are definitions for certain terms used herein to describe the present invention. These definitions apply to the terms as they are used throughout this specification, unless otherwise clearly indicated.

As used herein the singular forms “a”, “and”, and “the” include plural referents unless the context dearly dictates otherwise. For example, “a compound” refers to one or more of such compounds, while “the protein” includes a particular protein as well as other family members and equivalents thereof as known to those skilled in the art.

Interferon-γ. The nucleic acid sequences encoding IFN-γ polypeptides may be accessed from public databases, e.g. Genbank, journal publications, etc. While various mammalian IFN-γ polypeptides are of interest, for the treatment of human disease, generally the human protein will be used. Human IFN-γ coding sequence may be found in Genbank, accession numbers X13274; V00543; and NM_000619. The corresponding genomic sequence may be found in Genbank, accession numbers J00219; M37265; and V00536. See, for example. Gray et al. (1982) Nature 295:501 (Genbank X13274); and Rinderknecht et al. (1984) J.B.C. 259:6790.

The IFN-γ to be used in the compositions of the present invention may be any of natural IFN-γs, recombinant IFN-γs and the derivatives thereof so far as they have a IFN-γ activity, particularly human IFN-γ activity. Human IFN-γ exhibits the antiviral and anti-proliferative properties characteristic of the interferons, as well as a number of other immunomodulatory activities, as is known in the art. Although IFN-γ is based on the sequences as provided above, the production of the protein and proteolytic processing can result in processing variants thereof. The unprocessed sequence provided by Gray et al., supra. consists of 166 amino acids. Although the recombinant IFN-γ produced in E. coli was originally believed to be 146 amino acids, (commencing at amino acid 20) it was subsequently found that native human IFN-γ is cleaved after residue 23, to produce a 143 aa protein, or 144 aa if the terminal methionine is present, as required for expression in bacteria. During purification, the mature protein can additionally be cleaved at the C terminus after reside 162 (referring to the Gray et al. sequence), resulting in a protein of 139 amino acids, or 140 amino acids if the initial methionine is present, e.g. if required for bacterial expression. The N-terminal methionine is an artifact encoded by the mRNA translational “start” signal AUG which, in the particular case of E. coil expression is not processed away. In other microbial systems or eukaryotic expression systems, methionine may be removed.

For use in the subject methods, any of the native IFN-γ peptides, modifications and variants thereof, or a combination of one or more peptides may be used. IFN-γ peptides of interest include fragments, and can be variously truncated at the carboxy terminal end relative to the full sequence. Such fragments continue to exhibit the characteristic properties of human gamma interferon, so long as amino acids 24 to about 149 (numbering from the residues of the unprocessed polypeptide) are present. Extraneous sequences can be substituted for the amino acid sequence following amino acid 155 without loss of activity. See, for example, U.S. Pat. No. 5,690,925, herein incorporated by reference. Native IFN-γ moieties include molecules variously extending from amino acid residues 24-150; 24-151, 24-152; 24-153, 24-155; and 24-157. Any of these variants, and other variants known in the art and having IFN-γ activity, may be used in the present methods.

The sequence of the IFN-γ polypeptide may be altered in various ways known in the art to generate targeted changes in sequence. A variant polypeptide will usually be substantially similar to the sequences provided herein, i.e. will differ by at least one amino acid, and may differ by at least two but not more than about ten amino acids. The sequence changes may be substitutions, insertions or deletions. Scanning mutations that systematically introduce alanine, or other residues, may be used to determine key amino acids. Specific amino acid substitutions of interest include conservative and non-conservative changes. Conservative amino acid substitutions typically include substitutions within the following groups: (glycine, alanine); (valine, isoleucine, leucine); (aspartic add, glutamic acid); (asparagine, glutamine); (serine, threonine); (lysine, arginine); or (phenylalanine, tyrosine).

Modifications of interest that do not alter primary sequence include chemical derivatization of polypeptides, e.g., acetylation, or carboxylation. Also included are modifications of glycosylation, e.g. those made by modifying the glycosylation patterns of a polypeptide during its synthesis and processing or in further processing steps; e.g. by exposing the polypeptide to enzymes that affect glycosylation, such as mammalian glycosylating or deglycosylating enzymes. Also embraced are sequences that have phosphorylated amino acid residues, e.g. phosphotyrosine, phosphoserine, or phosphothreonine.

Included in the subject invention are polypeptides that have been modified using ordinary chemical techniques so as to improve their resistance to proteolytic degradation, to optimize solubility properties, or to render them more suitable as a therapeutic agent. For examples, the backbone of the peptide may be cyclized to enhance stability (see Friedler et al. (2000) J. Biol. Chem. 275:23783-23789). Analogs may be used that include residues other than naturally occurring L-amino acids, e.g. D-amino acids or non-naturally occurring synthetic amino acids. The protein may be pegylated to enhance stability.

The polypeptides may be prepared by in vitro synthesis, using conventional methods as known in the art, by recombinant methods, or may be isolated from cells induced or naturally producing the protein. The particular sequence and the manner of preparation will be determined by convenience, economics, purity required, and the like. If desired, various groups may be introduced into the polypeptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

B cell specific Antibodies: For the purposes of the present invention, antibodies of interest specifically bind to antigens present on B cells, particularly hyperproliferative B cells, e.g. B lineage lymphomas and leukemias, and the like. The term “antibody” is used in the broadest sense and specifically covers monodonal antibodies (including full length monoclonal antibodies), polyclonal antibodies, multispecific antibodies (e.g., bispecific antibodies), and antibody fragments so long as they exhibit the desired biological activity. Fragments comprise a portion of a full length antibody, generally the antigen binding or variable region thereof. Examples of antibody fragments include Fab, Fab′, F(ab′)₂, and Fv fragments; diabodies; linear antibodies; single-chain antibody molecules; and multispecific antibodies formed from antibody fragments. In some aspects of the invention, a combination of one or more antibodies with different specificities, either for epitopes of a single antigen, or for multiple antigens, may be used.

Markers that are specifically found on B cells include B220 epitope (CD45R), which is an exon specific epitope found on essentially all B cells, and is maintained throughout B cell development (Coffman et al. (1982) Immunol. Rev. 69:5-23). The B cell markers CD19, CD20; CD22; CD23 are selectively expressed on B cells and have been associated with B cell malignancies (Kalil and Cheson (2000) Drugs Aging 16(1):9-27; U.S. Pat. No. 6,183,744, herein incorporated by reference). Surface immunoglobulin, including epitopes present on the constant regions or idiotypic determinants, is a specific marker for B cells and has been utilized in immunotherapy (Caspar et al. (1997) Blood 90(9):3699-706). The MB-1 antigen is found on all normal immunoglobulin (Ig)-expressing cells, but not on T cells, thymocytes, granulocytes, or platelets, and expressed by virtually all Ig-expressing B cell tumors (Link et al. (1986) J Immunol 137(9):3013-8). Other B cell antigens of interest known to be expressed, for example, on non-Hodgkin's lymphomas, are Muc-1; B5; BB1; and T9 (Freedman et al. (1987) Leukemia 1(1):9-15).

Of particular interest is the CD20 antigen, also known as “Bp35”. CD20 is a human B cell marker that is expressed during early pre-B cell development and remains until plasma cell differentiation. The CD20 molecule may regulate a step in the activation process which is required for cell cycle initiation and differentiation, and is usually expressed at very high levels on neoplastic B cells. Thus, the CD20 surface antigen can be targeted for treating B cell lymphomas. U.S. Pat No. 5,736,137, herein incorporated by reference, describes the chimeric antibody “C2B8” that binds the CD20 antigen and its use to treat B cell lymphoma.

In a preferred embodiment, the antibody is a monoclonal antibody. Monoclonal antibodies are highly specific, being directed against a single antigenic site, and each monoclonal antibody is directed against a single determinant on the antigen. For example, the monoclonal antibodies to be used in accordance with the present invention may be made by the hybridoma method first described by Kohler et al. (1975) Nature 256:495, or may be made by recombinant DNA methods (see, e.g., U.S. Pat. No. 4,816,567). The monoclonal antibodies may also be isolated from phage antibody libraries using the techniques described in Clackson et al. (1991) Nature 352:624-628 (1991) and Marks et al. (1991) J. Mol. Biol. 222:581-597 (1991), for example. For clinical use, the monoclonal antibodies may be humanized forms of non-human antibodies. These are chimeric antibodies that contain sequences derived from both human and non-human immunoglobulin. For the most part, humanized antibodies are human immunoglobulins in which residues from a hypervariable region of the recipient are replaced by residues from a hypervariable region of a non-human species having the desired specificity, affinity, and capacity.

Specificity, as used herein, refers to the affinity of the antibody, and to the cross-reactivity with other antigens. In order to consider an antibody interaction to be “specific”, the affinity will be at least about 10⁻⁷ M, usually about 10^{-8 to -9} M, and may be up to 10⁻¹¹ or higher for the epitope of interest. It will be understood by those of skill in the art that the term “specificity” refers to such a high affinity binding, and is not intended to mean that the antibody cannot bind to other molecules as well. One may find cross-reactivity with different epitopes, due, e.g. to a relatedness of antigen sequence or structure, or to the structure of the antibody binding pocket itself. Antibodies demonstrating such cross-reactivity are still considered specific for the purposes of the present invention.

B cell hyperproliferative disorders. Hyperproliferative disorders, or malignancies, are conditions in which there is unregulated cell growth. The methods of the present invention are directed at hyperproliferative disorders that derive from cells in the B cell lineage, typically including hematopoietic progenitor cells expressing B lineage markers, pro-B cells, pre-B cells, B-cells and memory B cells; and that express markers typically found on such B lineage cells. For the purposes of the present invention, plasma cell malignancies will generally be excluded.

Of particular interest are non-Hodgkin's lymphomas (NHLs), which are a heterogeneous group of lymphoproliferative malignancies with differing patterns of behavior and responses to treatment. Like Hodgkin's disease, NHL usually originates in lymphoid tissues and can spread to other organs, however, NHL is much less predictable than Hodgkin's disease and has a far greater predilection to disseminate to extranodal sites. The NHLs can be divided into 2 prognostic groups: the indolent lymphomas and the aggressive lymphomas. Indolent NHL types have a relatively good prognosis, with median survival as long as 10 years, but they usually are not curable in advanced clinical stages. The aggressive type of NHL has a shorter natural history. A number of these patients can be cured with intensive combination chemotherapy regimens, but there is a significant number of relapses, particularly in the first 2 years after therapy.

Among the NHL are a variety of B-cell neoplasms, including precursor B-lymphoblastic leukemia/lymphoma; peripheral B-cell neoplasms, e.g. B-cell chronic lymphocytic leukemia; prolymphocytic leukemia; small lymphocytic lymphoma; mantle cell lymphoma; follide center cell lymphoma; marginal zone B-cell lymphoma; splenic marginal zone lymphoma; hairy cell leukemia; diffuse large B-cell lymphoma; Burkitt's lymphoma; high-grade B-cell lymphoma, (Burkfitt-like); etc.

Follicular lymphoma comprises 70% of the indolent lymphomas reported in American and European clinical trials. Most patients with follicular lymphoma are over age 50 and present with widespread disease at diagnosis. Nodal involvement is most common, often accompanied by splenic and bone marrow disease. The vast majority of patients with advanced stage follicular lymphoma are not cured with current therapeutic options, and the rate of relapse is fairly consistent over time, even in patients who have achieved complete responses to treatment Subtypes include follicular small cleaved cell (grade 1) and follicular mixed small cleaved and large cell (grade 2).

Marginal zone lymphomas were previously included among the diffuse small lymphocytic lymphomas. When marginal zone lymphomas involve the nodes, they are called monocytoid B-cell lymphomas, and when they involve extranodal sites (gastrointestinal tract, thyroid, lung, breast, skin), they are called mucosa-associated lymphatic tissue (MALT) lymphomas. Many patients have a history of autoimmune disease, such as Hashimoto's thyroiditis or Sjogren's syndrome, or of Helicobacter gastritis. Most patients present with stage I or II extranodal disease, which is most often in the stomach. When disseminated to lymph nodes, bone marrow, or blood, this entity behaves like other low-grade lymphomas. Large B-cell lymphomas of MALT sites are classified and treated as diffuse large cell lymphomas.

Splenic marginal zone lymphoma is an indolent lymphoma that is marked by massive splenomegaly and peripheral blood and bone marrow involvement, usually without adenopathy. This type of lymphoma is otherwise known as splenic lymphoma with villous lymphocytes, an uncommon variant of B-cell chronic lymphocytic leukemia. Management is similar to that of other low-grade lymphomas, but this lymphoma responds less well to chemotherapy that would ordinarily be effective for chronic lymphocytic leukemia.

Among the aggressive forms of NHL is diffuse large B-cell lymphoma, which is the most common of the non-Hodgkin's lymphomas, comprising 30% of newly-diagnosed cases. Most patients present with rapidly enlarging masses, often with symptoms both locally and systemically. Relapses after treatment are not uncommon, depending on the presence of various risk factors. Another aggressive form is follicular large cell lymphoma.

Lymphomatoid granulomatosis is an EBV positive large B-cell lymphoma with a predominant T-cell background. The histology shows association with angioinvasion and vasculitis, usually manifesting as pulmonary lesions or paranasal sinus involvement. Patients are managed like others with diffuse large cell lymphoma.

Primary mediastinal B-cell lymphoma is a subset of diffuse large cell lymphoma characterized by significant fibrosis on histology. Patients are usually female and young. Patients present with a locally invasive anterior mediastinal mass which may cause respiratory symptoms or superior vena cava syndrome. Therapy and prognosis are the same as for other comparably-staged patients with diffuse large cell lymphoma, except for advanced-stage patients with a pleural effusion, who have an extremely poor prognosis (progression-free survival is less than 20%) whether the effusion is cytologically positive or negative.

Mantle cell lymphoma is found in lymph nodes, the spleen, bone marrow, blood, and sometimes the gastrointestinal system (lymphomatous polyposis). Mantle cell lymphoma is characterized by CD5-positive follicular mantle B cells, a translocation of chromosomes 11 and 14, and an overexpression of the cyclin D1 protein. The median survival is significantly shorter (3-5 years) than that of other lymphomas, and this histology is now considered to be an aggressive lymphoma. A diffuse pattern and the blastoid variant have an aggressive course with shorter survival, while the mantle zone type may have a more indolent course. Refractoriness to chemotherapy is a usual feature

Lymphoblastic lymphoma is a very aggressive form of NHL. It often occurs in young patients, but not exclusively. It is commonly associated with large mediastinal masses and has a high predilection for disseminating to bone marrow and the central nervous system (CNS). Treatment is usually patterned after that for acute lymphoblastic leukemia (ALL). Since these forms of NHL tend to progress so quickly, combination chemotherapy is instituted rapidly once the diagnosis has been confirmed. Careful review of the pathologic specimens, bone marrow aspirate and biopsy specimen, cerebrospinal fluid cytology, and lymphocyte marker constitute the most important aspects of the pretreatment staging work-up.

Burkitt's lymphoma/diffuse small noncleaved cell lymphoma typically involves younger patients and represents the most common type of pediatric non-Hodgkin's lymphoma. These aggressive extranodal B-cell lymphomas are characterized by translocation and deregulation of the c-myc gene on chromosome 8. A subgroup of patients with dual translocation of c-myc and bcI-2 appear to have an extremely poor outcome despite aggressive therapy. Treatment of Burkitt's lymphoma/diffuse small noncleaved cell lymphoma involves aggressive multidrug regimens similar to those used for the advanced-stage aggressive lymphomas.

Patients who undergo transplantation of the heart, lung, liver, kidney, or pancreas usually require life-long immunosuppression. This may result in post-transplantation lymphoproliferative disorder, which appears as an aggressive lymphoma. Pathologists can distinguish a polyclonal B-cell hyperplasia from a monoclonal B-cell lymphoma; both are almost always associated with EBV. In some cases, usually for the polyclonal forms of the disease, withdrawal of immunosuppression results in eradication of the lymphoma. When this is unsuccessful or not feasible, a combination therapy is used. EBV-negative post-transplantation lymphoproliferative disorders occur late and have a particularly poor prognosis.

Chronic lymphocytic leukemia (CLL) is a disorder of morphologically mature but immunologically less mature lymphocytes and is manifested by progressive accumulation of these cells in the blood, bone marrow, and lymphatic tissues. Lymphocyte counts in the blood are usually equal to or higher than 10,000 per cubic millimeter. At present there is no curative therapy. CLL occurs primarily in middle-aged and elderly individuals, with increasing frequency in successive decades of life. The clinical course of this disease progresses from an indolent lymphocytosis without other evident disease to one of generalized lymphatic enlargement with concomitant pancytopenia. Complications of pancytopenia, including hemorrhage and infection, represent a major cause of death in these patients. Immunological aberrations, including Coombs-positive hemolytic anemia, immune thrombocytopenia, and depressed immunoglobulin levels may all complicate the management of CLL. CLL lymphocytes coexpress the B-cell antigens CD19 and CD20 along with the T-cell antigen CD5. CLL B cells express relatively low levels of surface-membrane immunoglobulin (compared with normal peripheral blood B cells) and a single light chain (kappa or lambda). CLL is diagnosed by an absolute increase in lymphocytosis and/or bone marrow infiltration coupled with the characteristic features of morphology and immunophenotype.

AIDS-related lymphomas are comprised of a narrow spectrum of histologic types consisting almost exclusively of B-cell tumors of aggressive type. These include diffuse large cell lymphoma; B-immunoblastic; and small non-cleaved, either Burkitt's or Burkitt's like. The HIV-associated lymphomas can be categorized into: primary central nervous system lymphoma (PCNSL); systemic lymphoma; and primary effusion lymphoma. All of these lymphomas differ from non-HIV-related lymphomas in their molecular characteristics, presumed mechanism of pathogenesis, treatment, and clinical outcome. All 3 pathologic types are equally distributed and represent aggressive disease. In general, the clinical setting and response to treatment of patients with AIDS-related lymphoma is very different from the non-HIV patients with lymphoma. The HIV-infected individual with aggressive lymphoma usually presents with advanced-stage disease that is frequently extranodal. The clinical course is more aggressive, and the disease is both more extensive and less responsive to chemotherapy. Immunodeficiency and cytopenias, common in these patients at the time of initial presentation, are exacerbated by the administration of chemotherapy. Therefore, treatment of the malignancy increases the risk of opportunistic infections that, in turn, further compromise the delivery of adequate treatment.

Acute lymphocytic leukemia (ALL) generally has an aggressive course, depending in part on the presence of the Philadelphia (Ph) chromosome. Patients with Ph chromosome-positive ALL are rarely cured with chemotherapy. Many patients who have molecular evidence of the bcr-abl fusion gene, which characterizes the Ph chromosome, have no evidence of the abnormal chromosome by cytogenetics.

Although the methods of the invention are primarily applied to NHL, in some cases treatment may be used in cases of Hodgkin's lymphoma, which is a lymphoma characterized by a pleomorphic lymphocytic infiltrate with malignant multinucleated giant cells. Most cases of Hodgkin's disease probably arise from germinal center B cells that are unable to synthesis immunoglobulin. The majority of cases in developing countries and about one third of those in the United States are associated with the presence of Epstein-Barr virus in the Reed-Sternberg cells. Treatment strategies depend on a number of factors including the presence of B symptoms, the histologic subtype, gender, and sexual maturity.

Pharmaceutical formulation. Preparations which are in such form as to permit the biological activity of the active ingredients to be unequivocally effective, and which contain no additional components which are toxic to the subjects to which the formulation would be administered. Pharmaceutically acceptable excipients (vehicles, additives) are those that can reasonably be administered to a subject to provide an effective dose of the active ingredient employed. Because B cell disorders are frequently found in the blood or lymph nodes, the route of administration for the subject combination therapy will preferably be parenteral; as used herein, the term “parenteral” includes intravenous, intramuscular, subcutaneous, rectal, vaginal or intraperitoneal administration.

A stable formulation is one in which the protein therein essentially retains its physical stability and/or chemical stability and/or biological activity upon storage. Various analytical techniques for measuring protein stability are available in the art. Stability can be measured at a selected temperature for a selected time period. Preferably, the formulation is stable at room temperature for at least 1 month and/or stable at about 2-8° C. for at least 1 year, preferably for at least 2 years. Furthermore, the formulation is preferably stable following freezing and thawing of the formulation.

The immunologically active B cell specific antibodies; and the gamma interferon will typically be formulated, usually separately, by standard technique within a pharmaceutically acceptable buffer, for example, sterile saline, sterile buffered water, propylene glycol, combinations of the foregoing, etc. Methods for preparing parenterally administrable agents are described in Pharmaceutical Carriers & Formulations, Martin, Remington's Pharmaceutical Sciences, 15th Ed. (Mack Pub. Co., Easton, Pa. 1975), which is incorporated herein by reference. More particularly, the compounds of the present invention can be formulated into pharmaceutical compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols.

In pharmaceutical dosage forms, the compounds may be administered in the form of their pharmaceutically acceptable salts. They may also be used in appropriate association with other pharmaceutically active compounds. The compounds can be formulated into preparations for injections by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The term “unit dosage form”, as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the unit dosage forms of the present invention depend on the particular compound employed and the effect to be achieved, and the pharmacodynamics associated with each compound in the host.

### METHODS OF USE

A combined therapy of IFN-γ and B cell specific antibodies is administered to a host suffering from a hyperproliferative B cell disorder. The compounds are administered at a combined effective dosage that over a suitable period of time substantially reduces the cellular proliferation, while minimizing any side-effects. It is contemplated that the composition will be obtained and used under the guidance of a physician for in vivo use.

The IFN-γ and antibodies can be delivered simultaneously, or within a short period of time, by the same or by different routes. In a preferred embodiment, the two components will be separately formulated. Part of the total dose may be administered by different routes.

Introduction of the antibodies and the IFN-γ in these dose ranges can be carried out as a single treatment or over a series of treatments. In a preferred embodiment, the IFN-γ is administered prior to the antibodies, usually at least about 1 week prior, and may be two to four weeks prior. In this way the immunomodulatory activity of the IFN-γ is initiated prior to the antibody treatment. The IFN-γ is then usually maintained during the course of antibody therapy. Other immunomodulatory agents that enhance ADCC may also be administered in combination with the IFN-γ, e.g. G-CSF, GM-CSF; IL-10 and TGF-β. With respect to the antibodies, it is preferred that dosing be carried out over a series of treatments; this preferred approach is predicated upon the treatment methodology associated with these diseases. While a single dosage provides benefits and can be effectively utilized for disease treatment/management, a preferred treatment course can occur over several stages; most preferably, being introduced to the patient once a week for between about 2 to 10 weeks, most preferably for about 4 weeks.

Effective dosages of the antibodies range from about 0.001 to about 30 mg/kg body weight, more preferably from about 0.01 to about 25 mg/kg body weight, and most preferably from about 0.4 to about 20.0 mg/kg body weight. Other dosages are viable; factors influencing dosage include, but are not limited to, the severity of the disease; previous treatment approaches; overall health of the patient; other diseases present, etc. The skilled artisan is readily credited with assessing a particular patient and determining a suitable dosage that falls within the ranges, or if necessary, outside of the ranges.

Effective dosages of IFN-γ range from about 0.5 μg/m² to about 500 μg/m², usually from about 1.5 μg/m², to 100 μg/m², depending on the size of the patient. This activity is based on 10⁶ international units (IU) per 50 μg of protein.

Those of skill will readily appreciate that dose levels can vary as a function of the specific compound, the severity of the symptoms and the susceptibility of the subject to side effects. Preferred dosages for a given compound are readily determinable by those of skill in the art by a variety of means. A preferred means is to measure the physiological potency of a given compound.

The susceptibility of a particular tumor cell to killing with the combined therapy may be determined by in vitro testing. For example, a culture of the tumor cell is combined with combinations at varying concentrations for a period of time sufficient to allow the active agents to induce cell killing. For in vitro testing, cultured cells from a biopsy sample of the tumor may be used. The viable cells left after treatment are then counted.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention which will be limited only by the appended claims.

All publications mentioned herein are incorporated herein by reference for the purpose of describing and disclosing, for example, the cell lines, constructs, and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

1. A method of treating a B cell hyperproliferative disorder, the method comprising:
administering to a patient an effective dose of an antibody that specifically binds to an antigen present on said B cells; and IFN-γ (gamma interferon).

2. The method according to claim 1, wherein said B cell hyperproliferative disease is a Non-Hodgkin's lymphoma.

3. The method according to claim 1, wherein said antibody is a monoclonal antibody.

4. The method according to claim 1, wherein said antibody is a humanized monoclonal antibody.

5. The method according to claim 1, wherein said antigen present on said B cells is CD20.

6. The method according to claim 1, wherein said antibody is administered at a dose of 0.001 to 30 μg/kg.

7. The method according to claim 1, wherein said IFN-γ is human IFN-γ.

8. The method according to claim 7, wherein said IFN-γ is administered at a dose of from 0.5 μg/m² to about 500 μg/m².

9. The method according to claim 7, wherein administration of said IFN-γ is initiated at least one week prior to initiation of treatment with said antibody.

10. A composition comprising:
an antibody that binds to an antigen present on B cells; and gamma interferon.

11. The composition of claim 10, wherein said antibody is a monoclonal antibody.

12. The composition of claim 10, wherein said antibody is an anti-CD20 antibody.

13. The composition of claim 10, wherein said antibody is in an amount sufficient to deliver a unit dose of about 0.001 to about 30 μg/kg.

14. The composition of claim 10, wherein said gamma interferon is human gamma interferon.

15. The composition of claim 10, wherein said gamma interferon is in an amount sufficient to deliver a unit does of about 0.5 micrograms/m² to about 500 micrograms/m².

16. Use of the composition of claim 10 as a medicament for the treatment of a B cell proliferative disorder.

17. Use according to claim 16, wherein the disorder is a Non-Hodgkin's lymphoma.
